SEQUENCE LISTING (8 sequences)

SEQ ID NO: 1 - DNA, 15 bp, Artificial Sequence
  chimera decoy NF-kappaB/STAT6-15mer-B
gggatttcctgggaa

SEQ ID NO: 2 - DNA, 10 bp, Artificial Sequence
  binding region of NF-kappaB
gggatttcct

SEQ ID NO: 3 - DNA, 11 bp, Artificial Sequence
  binding region of STAT6
ttcccaggaaa

SEQ ID NO: 4 - DNA, 10 bp, Artificial Sequence
  consensus sequence of NF-kappaB
gggrhtyyhc

SEQ ID NO: 5 - DNA, 10 bp, Artificial Sequence
  consensus sequence of STAT6
  misc_feature (4)..(7): n is a, c, g, or t
ttcnnnngaa

SEQ ID NO: 6 - DNA, 15 bp, Artificial Sequence
  chimera decoy NF-kappaB/STAT6-15mer-A
gggacttcccatgaa

SEQ ID NO: 7 - DNA, 10 bp, Artificial Sequence
  partial sequence of NF-kappaB/STAT6-15mer-A
gggacttccc

SEQ ID NO: 8 - DNA, 11 bp, Artificial Sequence
  partial sequence of NF-kappaB/STAT6-15mer-A
ttcatgggaag

(19) United States
(12) Patent Application Publication
(10) Pub. No.: US 2022/0072026 A1
(43) Pub. Date: Mar. 10, 2022

(54) TREATMENT OF SPINAL CONDITIONS WITH CHIMERA DECOY

(71) Applicants: The Regents of the University of California; Anges, Inc.

(72) Inventors: Koichi MASUDA, San Diego, CA (US); Takahiro NAKAZAWA, Osaka (JP)

(21) Appl. No.: 17/417,718

(22) PCT Filed: Dec. 24, 2019
(86) PCT No.: PCT/JP2019/050545

§ 371 Date: Jun. 23, 2021

Related U.S. Application Data
(60) Provisional application No. 62/784,599, filed on Dec. 24, 2018.

(51) Int. Cl. A61K 31/713; C12N 15/113; A61P 19/00

(52) U.S. Cl. CPC A61K 31/713; A61P 19/00; C12N 15/113

(57) ABSTRACT

Provided herein are methods and compositions for treating spinal conditions. In particular, use of double-stranded oligonucleotide decoys capable of binding to the DNA binding sites of two transcription factors (NF-κB and STAT6) for treatment of intervertebral disc degeneration, regenerating a chondrocyte extracellular matrix, spinal pain, and promoting synthesis of proteoglycan in intervertebral disc cells.

### CROSS REFERENCE TO RELATED APPLICATIONS

This application claims the priority benefit of U.S. Provisional Application No. 62/784,599, filed Dec. 24, 2018, which application is incorporated herein by reference.

### TECHNICAL FIELD

The invention relates generally to oligonucleotide decoys and more specifically to use of double-stranded oligonucleotide decoys capable of binding to the DNA binding sites of two transcription factors for treatment of spinal pain.

### BACKGROUND ART

Low back pain (LBP) is a major cause of disability in the United States today. Over a three-year period, 15-20% of Americans require medical care for back pain. The cost to society of back pain is over fifty (50) billion dollars because of lost productivity and medical care expenses. LBP can be caused by many components of the lumbar spine, particularly the intervertebral discs (IVDs), the paravertebral muscles, and the zygapophyseal, or facet joint.

Recent studies have suggested that up to 30% of LBP is generated by the facet joint. Although it has recently been suggested that facet joint pathology causes LBP, historically, degeneration of the IVD has been associated with back pain and it is thought that IVD degeneration precedes facet joint osteoarthritis (OA). However, the relationship between degenerative changes in the facet joint and the IVD are largely unknown.

Currently, most treatments of facet joint OA are limited to physical therapy, medial branch block, intraarticular local anesthesia, steroid injection or radiofrequency denervation. Based on the US Preventive Service Task Force (USPSTF) criteria, the evidence level for medial branch block is I˜II-1. The evidence levels for intraarticular facet joint blocks and radiofrequency denervation are II-1˜2 and II-2˜II-3, respectively. Surgery is also occasionally performed, although there is no clear evidence to support surgical intervention. Recently, the effects of hyaluronic acid for facet joint arthropathy were investigated, but the efficacy of this treatment remains controversial.

Inflammatory features are known to play a pivotal role in the progression of facet joint degeneration and pain generation [2, 3]. A one year follow-up of a double-blinded, controlled clinical trial that used lumbar facet joint nerve blocks to treat chronic LBP generated in the facet joint suggests that non-surgical therapies can be used to manage these patients. Facet joint injection is a commonly used minimally invasive procedure that involves an injection of a corticosteroid; however, steroids are known to have side effects, such as suppression of matrix synthesis by chondrocytes [4] and infection.

Accordingly, a need exists for a non-invasive therapy for treating LBP, such as facet joint pain.

### SUMMARY OF INVENTION

The present invention is based on the finding that chimera decoy oligodeoxynucleotide injection into facet joints showed similar or superior efficacy to dexamethasone in ameliorating facet joint pain induced by thrombin in rats. Accordingly, the invention provides a method for the treatment of spinal pain. The method includes comprising administering to a subject in need thereof an effective amount of a double-stranded oligonucleotide decoy capable of binding to the DNA binding site of NF-κB and to the DNA binding site of signal transducer and activator of transcription 6 (STAT6), thereby treating spinal pain in the subject. In various embodiments, the decoy has a size of from 13 mer to 15 mer. In various embodiments, the decoy has a sequence represented by SEQ ID NO: 1 or 6. In various embodiments, at least a part of bonds between each nucleotide in the double-stranded oligonucleotide decoy includes a phosphorothioate bond. In various embodiments, at least a part of bonds between each nucleotide in the double-stranded oligonucleotide decoy includes a phosphorothioate bond. In various embodiments, the 5′ end of the decoy is bound, via a linker or directly, to a PLGA nanoparticle. In various embodiments, the decoy is administered directly into a facet joint of the subject. In various embodiments, the decoy is administered via intradiscal injection or epidural injection. In similar aspects, the invention provides a therapeutic agent for spinal pain, comprising a double-stranded oligonucleotide decoy capable of binding to the DNA binding site of NF-κB and to the DNA binding site of signal transducer and activator of transcription 6 (STAT6); and a double-stranded oligonucleotide decoy capable of binding to the DNA binding site of NF-κB and to the DNA binding site of signal transducer and activator of transcription 6 (STAT6) for use in the treatment of spinal pain. The above descriptions with respect to the method for the treatment of spinal pain are equally applied to these similar aspects.

In another aspect, the invention provides a method of treating intervertebral disc degeneration in a subject. The method includes administering to a subject in need thereof an effective amount of a double-stranded oligonucleotide decoy capable of binding to the DNA binding site of NF-κB and to the DNA binding site of signal transducer and activator of transcription 6 (STAT6), thereby treating intervertebral disc degeneration in the subject. In various embodiments, the decoy has a size of from 13 mer to 15 mer. In various embodiments, the decoy has a sequence represented by SEQ ID NO: 1 or 6. In various embodiments, at least a part of bonds between each nucleotide in the double-stranded oligonucleotide decoy includes a phosphorothioate bond. In various embodiments, at least a part of bonds between each nucleotide in the double-stranded oligonucleotide decoy includes a phosphorothioate bond. In various embodiments, the 5′ end of the decoy is bound, via a linker or directly, to a PLGA nanoparticle. In various embodiments, the decoy is administered directly into a facet joint of the subject. In various embodiments, the decoy is administered via intradiscal injection or epidural injection. In similar aspects, the invention provides a therapeutic agent for intervertebral disc degeneration, comprising a double-stranded oligonucleotide decoy capable of binding to the DNA binding site of NF-κB and to the DNA binding site of signal transducer and activator of transcription 6 (STAT6); and a double-stranded oligonucleotide decoy capable of binding to the DNA binding site of NF-κB and to the DNA binding site of signal transducer and activator of transcription 6 (STAT6) for use in the treatment of intervertebral disc degeneration. The above descriptions with respect to the method of treating intervertebral disc degeneration in a subject are equally applied to these similar aspects.

In another aspect, the invention provides a method for regenerating a chondrocyte extracellular matrix in a subject. The method includes administering to a subject in need thereof an effective amount of a double-stranded oligonucleotide decoy capable of binding to the DNA binding site of NF-κB and to the DNA binding site of signal transducer and activator of transcription 6 (STAT6), thereby regenerating a chondrocyte extracellular matrix in the subject. In various embodiments, the decoy has a size of from 13 mer to 15 mer. In various embodiments, the decoy has a sequence represented by SEQ ID NO: 1 or 6. In various embodiments, at least a part of bonds between each nucleotide in the double-stranded oligonucleotide decoy includes a phosphorothioate bond. In various embodiments, at least a part of bonds between each nucleotide in the double-stranded oligonucleotide decoy includes a phosphorothioate bond. In various embodiments, the 5′ end of the decoy is bound, via a linker or directly, to a PLGA nanoparticle. In various embodiments, the decoy is administered directly into a facet joint of the subject. In various embodiments, the decoy is administered via intradiscal injection or epidural injection. In similar aspects, the invention provides an agent for regenerating a chondrocyte extracellular matrix, comprising a double-stranded oligonucleotide decoy capable of binding to the DNA binding site of NF-κB and to the DNA binding site of signal transducer and activator of transcription 6 (STAT6); and a double-stranded oligonucleotide decoy capable of binding to the DNA binding site of NF-κB and to the DNA binding site of signal transducer and activator of transcription 6 (STAT6) for use in the regeneration of a chondrocyte extracellular matrix. The above descriptions with respect to the method for regenerating a chondrocyte extracellular matrix in a subject are equally applied to these similar aspects.

In another aspect, the invention provides a method for promoting the synthesis of proteoglycan in intervertebral disc cells of a subject. The method includes administering to a subject in need thereof an effective amount of a double-stranded oligonucleotide decoy capable of binding to the DNA binding site of NF-κB and to the DNA binding site of signal transducer and activator of transcription 6 (STAT6), thereby promoting the synthesis of proteoglycan in intervertebral disc cells of the subject. In various embodiments, the decoy has a size of from 13 mer to 15 mer. In various embodiments, the decoy has a sequence represented by SEQ ID NO: 1 or 6. In various embodiments, at least a part of bonds between each nucleotide in the double-stranded oligonucleotide decoy includes a phosphorothioate bond. In various embodiments, at least a part of bonds between each nucleotide in the double-stranded oligonucleotide decoy includes a phosphorothioate bond. In various embodiments, the 5′ end of the decoy is bound, via a linker or directly, to a PLGA nanoparticle. In various embodiments, the decoy is administered directly into a facet joint of the subject. In various embodiments, the decoy is administered via intradiscal injection or epidural injection. In similar aspects, the invention provides an agent for promoting the synthesis of proteoglycan in intervertebral disc cells of a subject, comprising a double-stranded oligonucleotide decoy capable of binding to the DNA binding site of NF-κB and to the DNA binding site of signal transducer and activator of transcription 6 (STAT6); and a double-stranded oligonucleotide decoy capable of binding to the DNA binding site of NF-κB and to the DNA binding site of signal transducer and activator of transcription 6 (STAT6) for use in the promotion of the synthesis of proteoglycan in intervertebral disc cells of a subject. The above descriptions with respect to the method for promoting the synthesis of proteoglycan in intervertebral disc cells of a subject are equally applied to these similar aspects.

### BRIEF DESCRIPTION OF DRAWINGS

FIG. 1 is a graphical diagram showing the results from evaluation of mechanical allodynia in rats using the 50% paw withdrawal threshold response to mechanical stimulation by von Frey hair filaments of both injected and contralateral hind paws.

FIGS. 2A and 2B are graphical diagrams showing the results from behavioral assessment for general condition and activity caused by facet joint pain. To evaluate the general condition of rats, change in body weight (FIG. 2A) was analyzed. To assess changes in general activities caused by facet joint pain, a commercially available device consisting of video cameras and behavior analysis software (HOMECAGESCAN, Clever Sys Inc, Reston, Va.) was performed before surgery and 1 week after surgery (pre- and post-op) (FIG. 2B).

FIGS. 3A and 3B are graphical diagrams showing immunohistochemistry data of dorsal root ganglia for Iba- and CGRP (FIG. 3A) and a correlation of the immunohistochemistry staining and von Frey test results (FIG. 3B).

FIG. 4 is a graphical diagram showing the results of proteoglycan turnover in human intervertebral disc cells stimulated by interleukin-1.

FIG. 5 shows S-PG remaining in the tissue (ratio to control).

FIG. 6 shows the disc height index.

FIG. 7 shows MRI T2 spin-echo weight images.

FIG. 8 shows T2 MRI (L3/4 control, L2/3, L4/5 injected).

### MODE FOR CARRYING OUT THE INVENTION

The present invention provides that a chimeric decoy can be used in various embodiments to treat intervertebral disc degeneration to regenerate chondrocyte extracellular matrix, to promote synthesis of proteoglylcan, and to treat spinal pain.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

The term “comprising,” which is used interchangeably with “including,” “containing,” or “characterized by,” is inclusive or open-ended language and does not exclude additional, unrecited elements or method steps. The phrase “consisting of” excludes any element, step, or ingredient not specified in the claim. The phrase “consisting essentially of” limits the scope of a claim to the specified materials or steps and those that do not materially affect the basic and novel characteristics of the claimed invention. The present disclosure contemplates embodiments of the invention compositions and methods corresponding to the scope of each of these phrases. Thus, a composition or method comprising recited elements or steps contemplates particular embodiments in which the composition or method consists essentially of or consists of those elements or steps.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

All publications, patents, and patent applications mentioned in this specification are herein incorporated by reference to the same extent as if each individual publication, patent, or patent application was specifically and individually indicated to be incorporated by reference.

As used herein, the term “decoy” refers to a structure that resembles one to which a certain substance should originally bind or act. As provided herein, a decoy used for a transcription factor may be a double-stranded oligonucleotide having the same DNA sequence as the binding region of the transcription factor on a genome gene. In the presence of such an oligonucleotide decoy, some of the transcription factor molecules bind to the decoy oligonucleotide, instead of binding to the binding region on the genomic gene to which it should have bound. This results in a decrease in the number of transcription factor molecules that bind to the binding region on the genomic gene to which it should have bound, leading to a decrease in the activity of the transcription factor. A chimeric decoy contains DNA sequences for binding more than one transcription factor.

As used herein, the term “allodynia” refers to central pain sensitization (increased response of neurons) following normally non-painful, often repetitive, stimulation. Allodynia can lead to the triggering of a pain response from stimuli which do not normally provoke pain.

As used herein, “discogenic pain” refers to pain originating from a damaged vertebral disc, particularly due to degenerative disc disease.

Intervertebral discs (IVDs) of the spine consist of an outer annulus fibrosus (AF), which is rich in collagens that account for their tensile strength, and an inner nucleus pulposus (NP), which contains large proteoglycans (PGs) that retain water for resisting compression loading. Biologically, disc cells in both the AF and NP maintain a balance between anabolism and catabolism, or steady state metabolism, of their extracellular matrices (ECMs), and are modulated by a variety of substances including cytokines, enzymes, their inhibitors and growth factors such as insulin like growth factor (IGF), transforming growth factor β (TGF-β), and bone morphogenetic proteins (BMPs). Various enzymes, such as matrix metalloproteinases (MMPs), and cytokines, mediate the catabolic process, or breakdown of the matrix. The degeneration of an IVD is thought to result from an imbalance between the anabolic and catabolic processes, or the loss of steady state metabolism, that are maintained in the normal disc.

As used herein, “facet joints” refer to the joints in a spine that make the back flexible and enable a subject to bend and twist. Nerves exit the spinal cord through these joints on their way to other parts of the body. Healthy facet joints have cartilage, which allows the vertebrae to move smoothly against each other without grinding.

The term “subject” or “host organism,” as used herein, refers to any individual or patient to which the subject methods are performed. Generally the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

The term “therapeutically effective amount” or “effective amount” means the amount of a compound or pharmaceutical composition that elicits the biological or medical response of a tissue, system, animal or human that is being sought by the researcher, veterinarian, medical doctor or other clinician. Thus, the term “therapeutically effective amount” is used herein to denote any amount of a formulation that causes a substantial improvement in a disease condition when applied to the affected areas repeatedly over a period of time. The amount varies with the condition being treated, the stage of advancement of the condition, and the type and concentration of formulation applied. Appropriate amounts in any given instance will be readily apparent to those skilled in the art or capable of determination by routine experimentation.

As used herein, the terms “reduce” and “inhibit” are used together because it is recognized that, in some cases, a decrease can be reduced below the level of detection of a particular assay. As such, it may not always be clear whether the expression level or activity is “reduced” below a level of detection of an assay, or is completely “inhibited.” Nevertheless, it will be clearly determinable, following a treatment according to the present methods.

As used herein, “treatment” or “treating” means to administer a composition to a subject or a system with an undesired condition. The condition can include a condition, disease or disorder. “Prevention” or “preventing” means to administer a composition to a subject or a system at risk for the condition. The condition can include a predisposition to a disease or disorder. The effect of the administration of the composition to the subject (either treating and/or preventing) can be, but is not limited to, the cessation of one or more symptoms of the condition, a reduction or prevention of one or more symptoms of the condition, a reduction in the severity of the condition, the complete ablation of the condition, a stabilization or delay of the development or progression of a particular event or characteristic, or minimization of the chances that a particular event or characteristic will occur.

The terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, α-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As used herein, the term “gene” means the deoxyribonucleotide sequences comprising the coding region of a structural gene. A “gene” may also include non-translated sequences located adjacent to the coding region on both the 5′ and 3′ ends such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into heterogenous nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

A “sequence” of a nucleic acid refers to the order and identity of nucleotides in the nucleic acid. A sequence is typically read in the 5′ to 3′ direction. The terms “identical” or percent “identity” in the context of two or more nucleic acid or polypeptide sequences, refer to two or more sequences or subsequences that are the same or have a specified percentage of amino acid residues or nucleotides that are the same, when compared and aligned for maximum correspondence, e.g., as measured using one of the sequence comparison algorithms available to persons of skill or by visual inspection. Exemplary algorithms that are suitable for determining percent sequence identity and sequence similarity are the BLAST programs, which are described in, e.g., Altschul et al. (1990) “Basic local alignment search tool” J. Mol. Biol. 215:403-410, Gish et al. (1993) “Identification of protein coding regions by database similarity search” Nature Genet. 3:266-272, Madden et al. (1996) “Applications of network BLAST server” Meth. Enzymol. 266:131-141, Altschul et al. (1997) ““Gapped BLAST and PSI-BLAST: a new generation of protein database search programs” Nucleic Acids Res. 25:3389-3402, and Zhang et al. (1997) “PowerBLAST: A new network BLAST application for interactive or automated sequence analysis and annotation” Genome Res. 7:649-656, which are each incorporated by reference. Many other optimal alignment algorithms are also known in the art and are optionally utilized to determine percent sequence identity.

As used herein, the terms “functionally linked” and “operably linked” are used interchangeably and refer to a functional relationship between two or more DNA segments, in particular gene sequences to be expressed and those sequences controlling their expression. For example, a promoter/enhancer sequence, including any combination of cis-acting transcriptional control elements is operably linked to a coding sequence if it stimulates or modulates the transcription of the coding sequence in an appropriate host cell or other expression system. Promoter regulatory sequences that are operably linked to the transcribed gene sequence are physically contiguous to the transcribed sequence.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

As used herein “pharmaceutically acceptable carrier” encompasses any of the standard pharmaceutical carriers, such as a phosphate buffered saline solution, water and emulsions such as an oil/water or water/oil emulsion, and various types of wetting agents.

Various double-stranded oligonucleotide decoys showing binding affinity for a transcription factor are known for treating or preventing diseases caused by a transcription factor by administering a decoy for the transcription factor to reduce the activity of the transcription factor of interest. Recently, a double-stranded oligonucleotide decoy that includes a first binding site for a first transcription factor and a second binding site for a second transcription factor have been described (see, e.g., US Pub. No. 2018/0298381 and Intl. Pub. WO2017/043639, both of which are incorporated herein by reference). Briefly, a first strand including the sense strand of the first binding site and a second strand including the sense strand of the second binding site are hybridized to form a double strand. Further, the sense strand of the first binding site and the sense strand of the second binding site are at least partly hybridized.

An exemplary double-stranded oligonucleotide decoy described in US Pub. No. 2018/0298381 is a NF-κB/STAT6-15mer-B decoy, which is represented by the following Formula I (SEQ ID NO: 1):

| first strand | [I] | |
|---|---|---|
| 5′GGGATTTCCTgggaa3′ | | |
| | {close oversize parenthesis} | spacer |
| 3′ccctAAAGGACCCTT5′ | | |

second strand

Thus, for the NF-κB/STAT6-15mer-B having the structure represented by the Formula [I], the first transcription factor is NF-κB, and the second transcription factor is STAT6. The first and second strands are complementary, and thus the second strand is a complementary strand of the first strand. In the first strand, the sequence GGGATTTCCT (SEQ ID NO: 2), which is designated by capital letters, is the binding site for NF-κB, and in the second strand, the sequence TTCCCAGGAAA (SEQ ID NO: 3), which is designated by capital letters (this sequence is written, in the Formula [I], with its 3′ end on the left since it is a complementary strand; and thus this sequence and the sequence represented in the Formula [I] are the same in fact though they are written in opposite directions), is the binding site for STAT6.

Consensus sequences of transcription factors are often represented by general formulae. The consensus sequence of NF-κB is GGGRHTYYHC (SEQ ID NO: 4) (wherein R, represents A or G, Y represents C or T, and H represents A, C or T), and the consensus sequence of STAT6 is TTCNNNNGAA (SEQ ID NO: 5) (wherein N represents A, G, T or C). Therefore, the binding site GGGATTTCCT (SEQ ID NO: 2) for NF-κB in the first strand is the same as the consensus sequence of NF-κB, except that only one base at the 3′ end mismatches with the consensus sequence of NF-κB. The binding site TTCCCAGGAAA (SEQ ID NO: 3) for STAT6 in the second strand contains the whole of the consensus sequence of STAT6. When describing a base sequence, the base sequence of the sense strand is described, though the binding site for and the consensus sequence of the transcription factor are double-stranded. Thus, the base sequences of the binding sites and the consensus sequences as described above are all the base sequences of the sense strands. Thus, the first strand contains the sense strand of the binding site for NF-κB, and the second strand contains the sense strand of the binding site for STAT6.

An additional exemplary oligonucleotide decoy NF-κB/STAT6-15mer-A) is provided as Formula II (SEQ ID NO: 6):

| first strand | [II] | |
|---|---|---|
| 5′GGGACTTCCCatgaa3′ | | |
| | {close oversize parenthesis} | spacer |
| 3′ccctGAAGGGTACTT5′ | | |

second strand

Thus, for the NF-κB/STAT6-15mer-B having the structure represented by the Formula [II], the first transcription factor is NF-κB, and the second transcription factor is STAT6. The first and second strands are complementary, and thus the second strand is a complementary strand of the first strand. In the first strand, the sequence GGGACTTCCC (SEQ ID NO: 7), which is designated by capital letters, is the binding site for NF-κB, and in the second strand, the sequence TTCATGGGAAG (SEQ ID NO: 8), which is designated by capital letters (this sequence is written, in the Formula [II], with its 3′ end on the left since it is a complementary strand; and thus this sequence and the sequence represented in the Formula [II] are the same in fact though they are written in opposite directions), is the binding site for STAT6.

The chimeric decoy of the present invention may be a simple double strand, or may be a hairpin or dumbbell (staple) decoy in which one or both ends of each strand are bound via a spacer. Hairpin and dumbbell decoys are preferred because of their higher stability. When comprehensively evaluating the binding activities to transcription factors and the stability, the hairpin decoy is most preferred. Method of making such harpin double stranded chimeric decoys and other nuclease resistance modifications are well-known as described in U.S. Publication No. 2018/0298381.

Although the role of cytokines on cartilage matrix metabolism and cartilage degeneration has been recognized in cartilage research, preliminary studies indicate that facet joint chondrocytes in non-surgical samples can produce significant amounts of the cytokines, IL-1 and TNF-α; this may be a phenomenon specific to the facet joint. Interestingly, the presence of cytokines was higher at early stages of degeneration (grades 2/3) than at advanced stages of degeneration (grades 4/5); this may suggest cytokines are involved in the progression of facet joint cartilage degeneration (2/3->4/5). Furthermore, cytokine blocking studies have shown that prostaglandin (PG) synthesis was suppressed by constitutively-expressed cytokines (see, e.g., U.S. Pat. No. 7,585,848, incorporated herein by reference).

In vitro study indicated that chimera decoy (NF-κB/STAT6) significantly suppressed the gene expression of cytokines by synovial explant from knee joints of osteoarthritis patients. Additional results indicated that the intraarticular injection of original decoy (AMG0101, Formula I) to facet joints attenuated allodynia in the rat thrombin induced facet joint arthritis model. These results suggested that a chimera decoy can reverse negative balance of facet cartilage homeostasis and to suppress pain induced by cytokine pathways.

Accordingly, the invention provides a method for the treatment of spinal pain. The method includes comprising administering to a subject in need thereof an effective amount of a double-stranded oligonucleotide decoy capable of binding to the DNA binding site of NF-κB and to the DNA binding site of signal transducer and activator of transcription 6 (STAT6), thereby treating spinal pain in the subject.

As demonstrated herein, the chimera decoy oligodeoxynucleotide (chimera decoy), developed to bind to both NF-kB and signal transducer and activator of transcription 6 (STAT6) binding sites, was shown to decrease the gene expression of pro-inflammatory cytokines and pain-related molecules by synovial tissues [5] and to slow proteoglycan turnover accelerated by IL-1β [6]. Because this chimera decoy does not suppress matrix synthesis or increase the risk of infection, the chimera decoy is a novel local therapeutic for facet joint pain. The inhibition of cytokine pathways by chimera decoy will reduce pain generation by blocking several NF-κB driven cytokine pathways, such as IL-1 and TNF. The present invention describes clinically relevant efficacy data for the effect of chimera decoy on facet joint pain, in comparison with those for dexamethasone, in a recently developed rat thrombin-induced facet joint pain model [7].

As described herein, chimera decoy and dexamethasone demonstrated similar, significant analgesic effects on mechanical allodynia; these pain outcome measures were supported by IHC data of pain-related molecules in DRGs. Importantly, only the chimera decoy showed a significant effect on post-operative general activity, i.e., travel distance and body weight changes (known to be a sensitive indicator of postoperative pain [10]). The significant increase in body weight, found even during high activity, further supports the safety and efficacy of chimera decoy. The strong correlation between the results of pain status obtained from the von Frey test and IHC staining suggests the importance of pain marker analysis in the DRG for afferent nerve pain generation as additional confirmation of the efficacy outcome. As such, facet pain generation induced by thrombin injection may be ameliorated by injection of chimera decoy, which may serve as a new therapeutic approach for facet joint pain.

The chimeric decoy of the present invention can be administered as it is, or can also be administered after it is conjugated with a substance constituting an appropriate drug delivery system (DDS). Examples of DDSs for an oligonucleotide include liposomes containing cationic substances, cell membrane permeable peptides, polymers containing them, and atelocollagen. Besides these, the chimeric decoy can also be conjugated to PLGA (polylactic acid/glycolic acid copolymer) nanoparticles for administration. PLGA nanoparticles are particles having a diameter of tens of nanometers to hundreds of nanometers composed of PLGA. When the chimeric decoy is conjugated to PLGA nanoparticles, the 5′ end of the first strand of the chimeric decoy is preferably conjugated to PLGA nanoparticles via a disulfide linker and an amino linker. This can be carried out, for example, by reacting a PLGA-NHS ester with the chimeric decoy to obtain a PLGA-conjugated chimeric decoy and further making it into a nano-sized particle by utilizing the Marangoni effect.

Following administration of the decoy, to determine the efficacy of the treatment, the present methods encompass determining or measuring the level of LBP. In some embodiments, the methods may involve determining or measuring the level of the intervertebral disorder before treatment in order to establish the amount of decoy needed to sufficiently treat the LBP in the subject. In various embodiments, the level of fibrocartilage degrading factors or their precursors, e.g., pro-enzymes, mRNA, etc., can be measured to ascertain the amount of fibrocartilage degradation. Generally, a fibrocartilage-degrading factor encompasses any compound that, when present, will lead to the degradation of fibrocartilage tissue in an intervertebral disc. The fibrocartilagedegrading factor can act directly on fibrochondrocytes or fibrocartilage tissue to cause degradation, affect a compound that directly degrades fibrocartilage tissue, or affect a modulator of a compound that degrades fibrocartilage tissue. Fibrocartilage degrading factors include enzymes that directly degrade the cartilage matrix as well as other chemicals that stimulate cartilage degradation, including cytokines such as IL-1. IL-1 appears to indirectly cause fibrocartilage degradation by at least upregulating matrix metalloproteinase activity. Non-limiting examples of methods of measuring fibrocartilage-degrading factors include measuring nitric oxide (NO) production, proteinase detection, or both.

Proteinases, which occupy a specific group of fibrocartilage degrading factors, can be detected in normal and pathological intervertebral discs. These proteinases include, but are not limited to, matrix metalloproteinases (MMPs) and members of the ADAMTS family. Fibrocartilage-degrading factors including proteinases can be detected by any method known in the art. These methods include Western Blot analysis, immunohistochemistry, detection of RNA transcripts, and zymography. The fibrocartilage or fibrochondrocytes from the intervertebral disc can be treated with a fibrochondroprotective agent before measurement of the fibrocartilage degrading factors. Detection can also be conducted before contact, after contact, or both of a fibrocartilage-degrading factor. In various embodiments, the fibrocartilage degrading factors will be natural factors.

The route of administration of the chimeric decoy is not particularly limited, but may be preferably parenteral administration such as intravenous administration, intramuscular administration, subcutaneous administration, dermal administration, or direct administration to the target organ or tissue. The dosage is appropriately selected depending on, for example, the target disease, symptoms of the patient, and the route of administration, but usually 0.1 to 10000 nmol, preferably 1 to 1000 nmol, and more preferably 10 to 100 nmol per day for an adult may be administered.

Pharmaceutical compositions of transcription factor inhibitory compounds such as the chimera decoy, can be prepared by mixing one or more transcription factor inhibitory compounds with pharmaceutically acceptable carriers, excipients, binders, diluents or the like, to therapeutically treat, reverse or ameliorate a variety of intervertebral disc disorders and/or LBP. A therapeutically effective dose refers to that amount of one or more transcription factor inhibitory compounds sufficient to result in amelioration of symptoms of the intervertebral disc disorder. An effective dose can also refer to the amount of one or more transcription factor inhibitor compounds sufficient to result in prevention of the intervertebral disc disorder and/or LBP. In some embodiments, the effective dose will only partially prevent the intervertebral disc disorder and/or LBP. In these cases, the disorder of the intervertebral disc, although it may still exist, will be less than the expected intervertebral disc disorder if no treatment had been given.

The pharmaceutical compositions can be manufactured by methods well known in the art such as conventional granulating, mixing, dissolving, encapsulating, lyophilizing, emulsifying or levigating processes, among others. In certain embodiments, the transcription factor inhibitory compounds can be administered in a local rather than a systemic fashion, such as injection as a sustained release formulation. In some embodiments, an effective amount of the transcription factor inhibitory compounds can be administered in any satisfactory physiological buffer such as a phosphate buffer solution (PBS) or in a 5% lactose solution to the pathological intervertebral disc. The dosage forms disclosed in the instant specification are given by way of example and should not be construed as limiting the instant invention.

The formulations of the transcription factor inhibitory compounds can be designed for to be short acting, fast releasing, long acting, and sustained-releasing as described below. Thus, the pharmaceutical formulations can also be formulated for controlled release or for slow release, such as being contained within a biodegradable matrix or carrier.

The transcription factor inhibitor/decoy in the instant compositions can also exist in micelles or liposomes, or some other encapsulated form, or can be administered in an extended release form to provide a prolonged storage and/or delivery effect. Therefore, the pharmaceutical formulations can be compressed into pellets or cylinders and implanted as stints. Such implants can employ known inert materials such as silicones and biodegradable polymers.

A therapeutically effective dose of a transcription factor inhibitor can vary depending upon the route of administration and dosage form. The exact dose is chosen by a physician in view of the condition of a patient to be treated. Doses and administration are adjusted to provide a sufficient level of the active portion, or to maintain a desired effect. Specific dosages can be adjusted depending on conditions of disease, the age, body weight, general health conditions, sex, and diet of the subject, dose intervals, administration routes, excretion rate, and combinations of drugs. A sustained action pharmaceutical composition may be administered repeatedly within a certain interval such as every 3 to 4 days, every week, or once per two weeks (bi-monthly), depending on the half-life and clearance rate of a specific preparation. Guidance for specific doses and delivery methods are provided in publications known in the art. Any of the above dosage forms containing effective amounts are well within the bounds of routine experimentation and therefore, well within the scope of the instant invention.

The present invention also provides kits for carrying out the methods described herein. The present kits can also include one or more reagents, buffers, media, proteins, analytes, labels, cells, computer programs for analyzing results, and/or disposable lab equipment, such as culture dishes or multi-well plates, in order to readily facilitate implementation of the present methods. Solid supports can include beads, culture dishes, multi-well plates and the like.

The following examples are intended to illustrate but not limit the invention.

### Example 1

Rat Thrombin-Induced Facet Joint Arthritis Model

In this study, a rat model of thrombin-induced facet joint arthritis was developed. Thrombin is a protein essential to the clotting cascade, but it also has the ability to cleave PGs as well as create fragments of fibronectin and other matrix components. Thrombin has also been shown to stimulate cytokines and proteases. The injection of thrombin induced cartilage degeneration, both due to PG loss by direct enzyme action, and by damage caused by the inflammatory process driven by matrix fragments as well. Importantly, this rat facet joint arthritis model has been shown to be associated with sensory motor dysfunction, allodynia and changes in gait.

Eighty female Sprague-Dawley rats (11 weeks, ˜220 g) were used for this study. Under general anesthesia, the right L4/5 facet joint was carefully exposed without damaging the capsule. To induce facet joint pain and test the efficacy of therapeutics, bovine thrombin (20 U/2 μL saline) (Thrombin group), or thrombin+dexamethasone (Dexa group) (20 U/5 μg/2 μl saline) or thrombin+chimera decoy (Chimera group) (20 U/10 μg/2 μl saline) was slowly injected into the facet joint space using a MS05 (5 μl) syringe with a 33/28G dual gauge needle (Ito Corporation, Japan). Half of the rats were sacrificed at day 10 (D10) and the other half were sacrificed at 4 weeks (4 W).

All data are expressed as mean±standard error (SE). The data were statistically analyzed using two way-repeated analysis of variance (ANOVA) for overall analyses or one-way ANOVA for comparisons at each time point. Pearson's correlation coefficient was used to evaluate the relationship between von Frey test results and IHC staining. P values less than 0.05 are regarded as statistically different.

### Example 2

Behavioral Assessment for Mechanical Allodynia

It was believed that facet joint injury would lead to in changes in DRG and/or spinal function yielding sensitization and events that underlie a facilitated state of pain processing. Mechanical allodynia was evaluated in rats using the 50% paw withdrawal threshold response to mechanical stimulation by von Frey hair filaments of both injected and contralateral hind paws [8]. The von Frey hair was presented perpendicular to the plantar surface with sufficient force to cause slight buckling against the paw, and held for approximately 6-8 seconds (sec). Withdrawal of the paw and flinching were considered positive responses. The von Frey test results of the injected side were used for analysis.

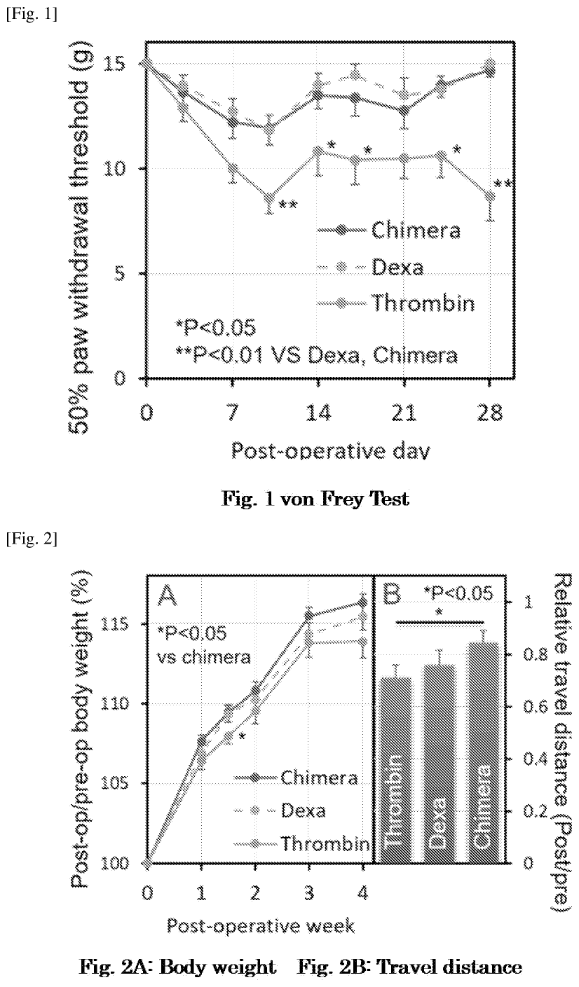

(FIG. 1):

Two-way repeated ANOVA revealed significant effects of time and treatment. Although withdrawal threshold was significantly reduced until day 10 after surgery in all groups (P<0.01), values in the Chimera and Dexa groups were significantly higher than that of the thrombin group (P<0.01). At day 10, withdrawal thresholds of the Dexa (11.83 g) and Chimera (11.91 g) groups were significantly higher than that of the Thrombin group (8.60 g) (P<0.01). These significant differences had been observed at most time points from day 10 to day 28 (P<0.05, One-way ANOVA).

To assess thermal nociceptive responses, a radiant heat source is focused on the plantar surface of the hindpaw. The time from the initiation of the radiant heat until paw withdrawal is measured (PWL). Rats are acclimatized for at least three consecutive days (baseline behavior testing) prior to starting an experiment. Behavioral testing is performed at the same time each day in a quiet dedicated room. Each paw is tested four times, alternating between paws with an interval of at least 1 minute between tests. The interval between two trials on the same paw is at least 5 minutes. A significant decrease in the PWL is defined as thermal hyperalgesia. A cut-off of 20 sec is employed to avoid tissue injury.

### Example 3

General Condition and Activity

To evaluate the general condition of rats, change in body weight was analyzed. To assess changes in general activities caused by facet joint pain, a commercially available device consisting of video cameras and behavior analysis software (HOMECAGESCAN, Clever Sys Inc, Reston, Va.) was utilized before surgery and 1 week after surgery (pre- and post-op).

The video camera was used to capture a movie of the gait using a mirror system to capture the position of each foot as the rat walks. The rats were allowed to acclimate to the unit for one week prior to starting the project. During this time they were trained to walk on the moving treadmill at a constant speed of 15 cm/sec for 20 sec. A high speed digital camera was positioned beneath the treadmill to monitor the movement of the rat, recording 100 frames/sec. Once the rats were accustomed to the treadmill, they were walked at 15 cm/sec for 20 sec, with a 60 sec break between sessions for the duration of the study. Results were analyzed using Clever Sys. Inc. software, looking at over 35 measures of gait. This system has been validated at assessing gait abnormalities in various disease states, including a rat model of OA.

(FIGS. 2A and 2B):

At day 10, body weight increase in the Chimera group (+9.66%) was significantly higher than that in the Thrombin group (6.72%, P<0.05). Travel distance was significantly decreased at one-week post-surgery in all groups (P<0.01). The relative travel distance (post op/pre-op) in the Chimera group was significantly higher than that in the Thrombin group (0.84 vs. 0.73, P<0.05).

### Example 4

Immunohistochemistry of DRGs

After sacrifice, the expressions of ionized calcium binding adaptor molecule-1 (Iba-1; a microglia/macrophage specific calcium binding protein), and calcitonin gene-related peptide (CGRP, a pain-related neuropeptide) were analyzed in DRG neurons of at least six rats in each group. Semi-quantitative analyses were conducted as previously published [9]. The correlation between von Frey test results and IHC staining was also assessed.

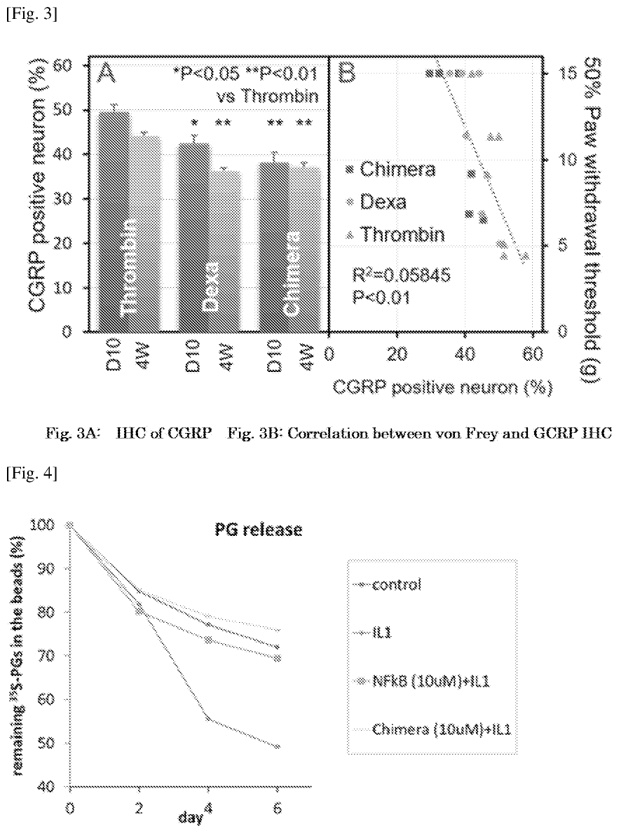

(FIGS. 3A and 3B): Iba-1: At the 4-week time point, the average number of Iba1-positive microglia/mm2 in the Chimera group was significantly lower than that in the Thrombin group (−33%, P<0.05). No significant differences were observed at day 10. CGRP: At day 10, the average percentage of CGRP-positive neurons in the Dexa and Chimera groups was significantly lower than that in the Thrombin group (−14.3%, P<0.05, −22.9%, P<0.01, respectively). At 4 weeks, these differences were maintained. Importantly, IHC data for Iba- and CGRP were negatively correlated with the von Frey test results (P<0.05, see FIG. 3B).

### Example 5

Chimera Decoy Modulates Degeneration of Human Intervertebral Disc Cells

A study to evaluate whether Chimera Decoy was able to modulate proteoglycan (PG) turnover in human intervertebral disc cells stimulated by interleukin-1 was performed.

Human nucleus pulposus (NP) cells from scoliotic spines of 16 year-old patients were expanded in monolayer culture, passaged and embedded in alginate beads, as previously reported [11]. Beads were cultured for 4 weeks to obtain a chondrocytic phenotype. Proteoglycans of the human NP cells embedded in the alginate beads were then pre-labeled with ³⁵S, washed and further cultured in the presence of IL-1β (5 ng/ml) with/without Chimera decoy (10 μM) or NF-κB decoy for up to 6 days. ³⁵S-PGs in the collected culture media (day 2, 4 and 6) and ³⁵S-PGs remaining in the beads after the 6-day culture period were measured and the percentage of remaining PGs of the total ³⁵S-PGs (media+beads) was calculated to reveal the degree of PG degradation.

The effect of Chimera Decoy on the rate of loss of proteoglycans from the beads was studied using the pulse-chase procedure. As shown in FIG. 4, treatment with IL-1β significantly accelerated the PG turnover of alginate beads (IL-1β, 49%, Control 72% on day 6, P<0.05, two-way ANOVA). On the other hand, the Chimera Decoy group reversed the effect of IL-1β (Chimera+IL1: 76% on day 6, P<0.05) and did not show a significant difference compared to the control group. Although the NF-κB decoy (10 μM) group showed a similar trend (NF-κB+IL-1 decoy; 69% on day 6), the difference did not reach a statistically significant level.

The results indicated that Chimera Decoy could counteract the acceleration of catabolism in human disc cells stimulated with IL-1. This suggests that Chimera Decoy can delay or reverse disc degeneration and, thus, reduce pain in disc degeneration patients.

The purpose of this study is to investigate the effects of chimera decoy ODN on PG degradation in human anulus fibrosus (AF) tissue from patients undergoing discectomy procedures.

### Example 6

The Effect of Chimera Decoy Oligodeoxynucleotide on Proteoglycan Degradation in Human Anulus Fibrosus Tissue

Materials and Methods:

The AF tissue was obtained from seven patients undergoing lumbar spine surgery [67±9 years-old; Male 6, Female 1]. The tissue was washed, cut into 3 mm pieces and incubated as explant cultures (three to five pieces in each group).

PG Turnover:

The AF tissue was pre-labeled with 20 μCi/ml ³⁵S for 16 hours as previously described. The explants were cultured in DMEM/F-12 media with 20% FBS in the presence or absence of NFkB decoy ODN (10 μM) or Chimera decoy ODN (10 μM) for up to 6 days. The media were replaced every other day with the same treatment media and the cultured media was collected. The tissue was collected at the end of the incubation period and digested with papain. The amount of ³⁵S-PGs in the media and digests was measured by a rapid Alcian blue filtration assay. The ³⁵S-PGs remaining over total ³⁵S-PGs synthesized was assessed. The data was also normalized by the control group of each patient.

Statistical Analyses:

Two-way ANOVA with Fisher PSLD test as a post hoc test. The results are expressed as mean±standard error.

Results:

The loss of PG in the control levels was significantly different in these 7 patients (Table 1, P<0.01). PG degradation in the chimera decoy ODN group was significantly suppressed compared to the control group (p=0.025). (Control, NFkB, Chimera; Day 2:1.00±0.01, 1.00±0.02, 1.04±0.02, Day 4: 1.00±0.02, 1.04±0.05, 1.15±0.07, Day 6: 1.00±0.05, 1.18±0.12, 1.34±0.13) (FIG. 5). After 6 days culture, the chimera group retained 34% more proteoglycan compared to the control. There was no significant difference in PG loss between the NFkB decoy group and the other groups.

Conclusion:

The addition of chimera decoy ODN to explant cultures of human AF from degenerated discs significantly suppressed PG loss compared to the control group. NFkB decoy, which inhibits only the NFkB pathway, showed a tendency to suppress PG loss, but was not significant in this experiment.

Table 1. PG is remaining on Day 6 (% of Labeled PG)

TABLE 1

| Patient | | | | | |
|---|---|---|---|---|---|
| Number | age | gender | Control | Decoy | Chimera |
| 1 | 52 | Male | 14.4% | 13.5% | 19.5% |
| 2 | 63 | Female | 64.5% | 62.6% | 47.8% |
| 3 | 72 | Male | 38.3% | 43.9% | 56.4% |
| 4 | 81 | Male | 51.4% | 59.0% | 69.8% |
| 5 | 62 | Male | 53.3% | — | 70.9% |
| 6 | 72 | Male | 44.9% | 34.7% | 40.0% |
| 7 | 66 | Male | 16.8% | 40.6% | 46.0% |

### Example 7

The Efficacy of Chimera Decoy on Intervertebral Disc Degeneration in the Rabbit Annular-Puncture Model

In this model, degenerated nucleus pulposus (NP) from rabbits is implanted as a xenograft tissue on the dorsal root ganglion (DRG) of RNU nude rats to determine if the tissues induce functional and sensory dysfunction.

The specific purpose of this experiment is to determine (1) if an intradiscal injection of Chimera decoy inhibits excessive expression of pro-inflammatory cytokines and matrix-degrading enzymes, and (2) if Chimera decoy intradiscal injections using varying doses into rabbit discs attenuates degenerated disc tissue-induced pain in nude rats.

Methods:

Rabbit annular-puncture disc degeneration model and the injection of Chimera decoy: Female New Zealand white rabbits (n=80) were used in this study. Under general anesthesia, the anulus fibrosus was punctured with an 18-gauge needle in two noncontiguous discs (L2/3 and L4/5). Four weeks after the initial puncture, either vehicle phosphate buffered saline (10 μl), Chimera decoy (10, 100 μg in 10 μl saline) or NFκB Decoy (100 μg in 10 μl saline) was injected into the center of the nucleus pulposus (NP) using a 26-gauge needle.

Radiographic Analysis of Disc Height:

Intervertebral disc (IVD) height was obtained from lateral radiographs and expressed as disc height index (DHI). The DHI was normalized to the preoperative DHI (% DHI) and further normalized to the DHI of the L3/4 non-punctured disc.

MRI Analyses:

The degeneration grade of IVDs was classified according to Pfirrman using T2 weighted images.

Results:

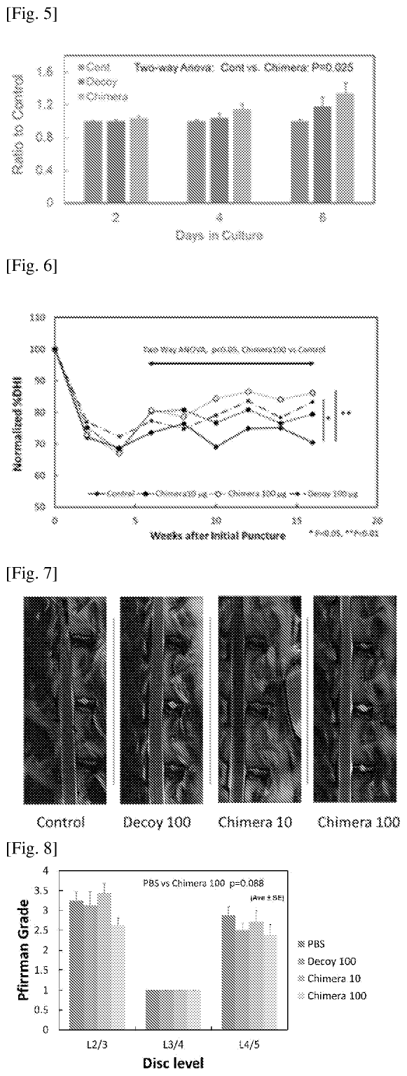

DHI (FIG. 6):

Repeated two-way ANOVA revealed that treatment significantly affected % DHI (P<0.05). Posthoc analysis indicated that % DHI in the Chimera 100 ug group was significantly higher than the PBS group. At week 16, the Chimera 100 ug and Decoy 100 g groups showed a significant increase, compared to the PBS group in % DHI (p<0.01 and p<0.05 respectively).

MRI T2 spin-echo weighted images (FIG. 7) and Pfirrmann Grade (FIG. 8).

The Pfirrmann grade tended to be lower in the Chimera 100 μg group compared to the PBS group (P<0.088).

Discussion:

The injection of Chimera decoy into the NP four weeks after annular-puncture restored disc height and slightly improved the MRI grade. The data indicated that a Chimera decoy injection can induce structural changes at 100 μg.

Chimera injection into discs reduced pain generation in the xenograft rat radiculopathy model. These results suggest that an injection of Chimera Deoy changes the pathological status of degenerated discs and reduces pain generation and, therefore, serves as a new therapeutic approach for degenerative disc disease.

Although the invention has been described with reference to the above example, it will be understood that modifications and variations are encompassed within the spirit and scope of the invention. Accordingly, the invention is limited only by the following claims.

### REFERENCES

[1] Sehgal, et al. Pain Physician. 2007; 213-28.
[2] Kim, et al. Osteoarthritis and Cartilage. 2015; 2242-51.
[3] Hemmad, et al. Trans Orthop Res Soc. 2010; 933.
[4] Wernecke, et al. Orthop J Sports Med. 2015; 2325967115581163.
[5] Miyazaki, et al. Trans Orthop Res Soc. 2018; 0387.
[6] Kato, et al. Trans Orthop Res Soc. 2017; 0175.
[7] Yamaguchi, et al. The International Society for the Study of the Lumbar Spine. 2013; SP19.
[8] Chaplan, et al. J Neurosci Methods. 1994; 55-63.
[9] Miyazaki, et al. Eur Spine J. 2018; 739-51.
[10] Brennan, et al. Lab animal. 2009; 87-93.
[11] Aota, et al. Differential effects of fibronectin fragment on proteoglycan metabolism by intervertebral disc cells: a comparison with articular chondrocytes. Spine (Phila Pa. 1976). 2005; 30(7):722-728.
[12] WO 2017/043639 A1

1. A method for any one of: (a) the treatment of intervertebral disc degeneration in a subject in need thereof, (b) regenerating a chondrocyte extracellular matrix in a subject in need thereof, (c) promoting the synthesis of proteoglycan in intervertebral disc cells of a subject in need thereof, or (d) the treatment of spinal pain a subject in need thereof, comprising administering to the subject an effective amount of a double-stranded oligonucleotide decoy capable of binding to the DNA binding site of NF-κB and to the DNA binding site of signal transducer and activator of transcription 6 (STAT6), thereby (a) treating the intervertebral disc degeneration in the subject, or (b) regenerating the chondrocyte extracellular matrix in the subject, or (c) promoting the synthesis of proteoglycan in the intervertebral disc cells of the subject, or (d) treating spinal pain in the subject, respectively.

2. The method of claim 1, wherein the decoy has a size of from 13 mer to 15 mer.

3. The method of claim 2, wherein the decoy comprises the sequence represented by SEQ ID NO: 1 or 6.

4. The method of claim 1, wherein at least a part of bonds between each nucleotide in the double-stranded oligonucleotide decoy comprises a phosphorothioate bond.

5. The method of claim 1, wherein the 5′ end of the decoy is bound, by a linker or directly, to a PLGA nanoparticle.

6. The method of claim 1, wherein the decoy is administered directly into a facet joint of the subject.

7. The method of claim 1, wherein the decoy is administered by intradiscal injection or epidural injection.

8. (canceled)

9. The method of claim 1, wherein the chondrocyte extracellular matrix is an intervertebral disc cell extracellular matrix.

10-15. (canceled)

16. The method of claim 1, wherein the intervertebral disc cells comprise nucleus pulposus cells or anulus fibrosus cells or both.

17-28. (canceled)

29. A therapeutic agent for intervertebral disc degeneration or spinal pain, comprising a double-stranded oligonucleotide decoy capable of binding to the DNA binding site of NF-κB and to the DNA binding site of signal transducer and activator of transcription 6 (STAT6).

30. (canceled)

31. An agent for regenerating a chondrocyte extracellular matrix or promoting the synthesis of proteoglycan in intervertebral disc cells of a subject, comprising a double-stranded oligonucleotide decoy capable of binding to the DNA binding site of NF-κB and to the DNA binding site of signal transducer and activator of transcription 6 (STAT6).

32-36. (canceled)
